(19) United States
(12) Patent Application Publication
(10) Pub. No.: US 2016/0029950 A1
(43) Pub. Date: Feb. 4, 2016

(54) METHOD AND APPARATUS FOR MEASURING ANESTHETIC DEPTH USING CEPSTRUM TECHNIQUE

(71) Applicant: CHARM ENGINEERING CO., LTD., Yongin-si, Gyeonggi-do (KR)

(72) Inventors: Ho-Jong CHANG, Daejeon (KR); Eung Hwi KIM, Daejeon (KR); Sang Hyun PARK, Gongju-Si, Chungcheongnam-Do (KR); Seung Kyun HONG, Seoul (KR); Kwang Moo KIM, Seongnam-Si, Gyeonggi-Do (KR)

(21) Appl. No.: 14/767,285

(22) PCT Filed: Feb. 13, 2014
(86) PCT No.: PCT/KR2014/001200

§ 371 Date: Aug. 11, 2015

(30) Foreign Application Priority Data
Feb. 14, 2013 (KR) 10-2013-0015676

(51) Int. Cl. A61B 5/00; A61B 5/04; A61B 5/048

(52) U.S. Cl. CPC A61B 5/4821; A61B 5/048; A61B 5/04014

(57) ABSTRACT

The present invention relates to a more accurate method for measuring anesthetic depth compared to existing methods for measuring anesthetic depth by using a cepstrum technique, thereby providing an anesthetic depth at an appropriate time even during sudden changes in anesthetic states. The method for measuring anesthetic depth using the cepstrum technique comprises the steps of: a first characteristic vector extraction portion receiving a first EEG signal as an input signal, calculating with a mel-frequency cepstral coefficient (MFCC), and extracting a first characteristic vector; a second characteristic vector extraction portion receiving, as input signals, a second EEG signal from an anesthetic state and a third EEG signal from a non-anesthetic state, calculating with the mel-frequency cepstral coefficient (MFCC), and extracting a second characteristic vector and a third characteristic vector; and a quantifying portion dividing, into a plurality of sections, an area between two axes of a vector flat surface having the second characteristic vector and the third characteristic vector as the two axes, and quantifying a position of the first characteristic vector within the plurality of sections to output an anesthetic depth index.

### TECHNICAL FIELD

The present invention relates to a method for measuring an anesthetic depth, and more particularly, to a method and an apparatus for measuring an anesthetic depth by using a cepstrum technique, capable of providing anesthetic depth information timely despite a rapid change in an anesthetic state by providing an accurate value of an anesthetic depth and improving a tracking speed.

### BACKGROUND ART

Generally, in the field of medical practice including an operation and a treatment, when pain is applied to a subject, neurotransmission is blocked through anesthesia so that the pain is removed or reduced. During an operation for a severe disease or symptom, general anesthesia is performed, and a patient under general anesthesia should be continuously observed. An anesthetic state of a patient should be checked by sensing an anesthetic depth. While an operation should be performed under sufficient anesthesia, there is a problem in that a patient suffers from mental pain due to awakening during the operation.

Accordingly, during an operation, an anesthetic depth should be continuously measured, and a method for observing a clinical aspect and a method for analyzing a bioelectric signal have been mainly used as a method for measuring an anesthetic depth. The method for analyzing a bioelectric signal includes a method for measuring and analyzing brainwaves so as to evaluate an effect of an anesthetic agent on the central nervous system, and there are also various kinds of monitoring apparatuses to which a method of using brainwaves is applied. The reason there are various kinds of anesthetic depth monitoring apparatuses using brainwaves is that the respective apparatuses have different algorithms for analyzing and evaluating brainwaves.

Currently, a bispectral index (hereinafter, referred to as “BIS”) analyzing apparatus is most popularly used as an anesthetic depth monitoring apparatus. The BIS analyzing apparatus is one of the apparatuses in which a brainwave-based anesthetic depth measuring technique is developed and adopted for the first time therein, displays an anesthetic depth as “BIS” to be digitized within a range of 0-100, and verifies the clinical reliability of BIS by comparing BIS with a conventional anesthetic depth measuring standard or with an index calculated in another anesthetic depth instrument.

With a conventional anesthetic depth monitoring apparatus including such as BIS analyzing apparatus, a user, who is an anesthetic depth clinical subject or an anesthetic depth monitor, is unable to improve or change brainwave analyzing algorithms of instruments, so that an algorithm suitable for patient's characteristics may not be applied and accordingly an anesthetic depth of the patient cannot be accurately monitored. Furthermore, since the details of analyzing algorithms installed in instruments are not disclosed, the apparatus is not suitable for a clinical anesthetic depth study and there are many difficulties in proving an algorithm error.

Moreover, an anesthetic depth monitoring apparatus such as the BIS analyzing apparatus has a problem in that an anesthetic state of a patient is unable to be rapidly sensed because a speed for tracking a rapid change in an anesthetic state is slow.

Patent Document 1 relates to a system and a method for measuring a brain activity and an anesthetic depth through a brainwave signal analysis, wherein values may be very accurately calculated compared to a conventional spectrum analysis, wavelet analysis, or entropy analysis, although the structure of a basic algorithm is very simple.

(Patent Document 1) Korean Patent Application Laid-open Publication No. 2012-0131027 (publicized on Dec. 4, 2012)

### DISCLOSURE OF THE INVENTION

### Technical Problem

To resolve the above-described problem, an object of the present invention is to provide a method for measuring an anesthetic depth by using a cepstrum technique, which provides an accurate anesthetic depth and rapid responses to a change in an anesthetic degree at a high tracking speed.

### Technical Solution

To resolve the above-described problem, a method for measuring an anesthetic depth by using a cepstrum technique according to the present invention includes: extracting, by a first feature vector extracting part, a first feature vector by receiving a first EEG signal as an input signal and by performing a mel frequency cepstral coefficient (MFCC) calculation; extracting, by a second feature vector, a second feature vector and a third feature vector by receiving a second EEG signal in an anesthetic state and a third EEG signal in a non-anesthetic state as input signals and performing an MFCC calculation; and outputting, by a quantifying part, an anesthetic depth index through a quantifying part by dividing, into multiple sections, an area between the second and third feature vectors which are both axes of a vector plane, and quantifying a position at which the feature vector is located, among the multiple sections.

According to a preferable embodiment of the present invention, the extracting of the first feature vector further includes performing at least one of wavelet transformation or low frequency band pass filtering on the first EEG signal during an operation to remove noise and select and output a signal having only a predetermined frequency range.

According to a preferable embodiment of the present invention, the outputting of anesthetic depth index includes scaling the quantified signal in an index ranging from 1 to 100 to be displayed on a screen display part.

According to a preferable embodiment of the present invention, the extracting of the first feature vector includes dividing the first EEG signal into sections by short time to perform Fourier transformation the divided signals for each section and then to sum up results.

According to a preferable embodiment of the present invention, the extracting of the first feature vector includes filtering the Fourier-transformed signals by a plurality of filter banks having different frequency bands and calculating a power spectrum for each of the signals.

According to a preferable embodiment of the present invention, the extracting of the first feature vector includes reducing signal distortion by a frequency by performing a log calculation on the power spectrum signals.

According to a preferable embodiment of the present invention, the extracting of the first feature vector includes extracting the first feature vector by selecting only a signal, which passes through a predetermined filter in the plurality of filter banks, from among the signals obtained after discrete cosine transformation performed on the signals obtained after the log calculation.

According to another embodiment of the present invention, an apparatus for measuring an anesthetic depth by using a cepstrum technique according to the present invention includes: a first feature vector extracting part configured to output a first feature vector by performing a mel frequency cepstral coefficient (MFCC) calculation on a first EEG signal; a second feature vector extracting part configured to output a second feature vector and a third feature vector by performing an MFCC calculation on a second EEG signal in an anesthetic state and a third EEG signal in a non-anesthetic state; and a quantifying part configured to output an anesthetic depth index by dividing, into multiple sections, an area between the second and third feature vectors which are both axes of a vector plane, and quantifying a position at which the first feature vector is located, among the multiple sections.

According to a preferable embodiment of the present invention, the first feature vector extracting part further includes a noise removing configured to perform wavelet transformation and low frequency band pass filtering on the first EEG signal.

According to a preferable embodiment of the present invention, the first feature vector extracting part further includes a local Fourier transforming part which divides the first EEG signal into sections by short time to perform Fourier transformation on each of the sections.

According to a preferable embodiment of the present invention, the first feature vector extracting part further includes a mel filter bank which includes a plurality of filters, having different central frequencies and frequency bands overlapping each other for predetermined sections, and receives an output of the local Fourier transforming part as an input signal to filter the received signal.

According to a preferable embodiment of the present invention, the first feature vector extracting part further includes a log calculating part for reducing signal distortion by a frequency by performing a log calculation on the signals filtered from the mel filter bank.

According to a preferable embodiment of the present invention, the first feature vector extracting part further includes a discrete cosine transforming part for performing discrete cosine transformation (DCT) on the signals obtained after the log calculation.

According to a preferable embodiment of the present invention, the first feature vector extracting part further includes a coefficient extracting part configured to select only a signal, which passes through a predetermined filter in the filters of the mel filter bank, from among output signals of the discrete cosine transforming part, and outputting the selected signal to the first feature vector.

According to a preferable embodiment of the present invention, a scaling part for scaling the output of the quantifying part in an index ranging from 1 to 100 is further included.

According to a preferable embodiment of the present invention, an error removing part configured to represent the output of the first feature vector extracting part as a histogram and select a past output value for a signal outside an error range to be outputted as a weighted average value is further included.

### Advantageous Effects

According to the present invention, an anesthetic depth may be accurately analyzed by using an accessing technique completely differing from a conventional anesthetic depth analyzing algorithm and considering frequency characteristics.

According to the present invention, a real-time process may be easily performed due to a simple algorithm, thereby more accurately capturing a change in a state during anesthesia.

A conventional problem that when an anesthetic degree is rapidly changed, a reaction speed is low because of a low tracking speed of conventional BIS technique, is solved, and therefore a change in a state of from an awakening state to an anesthetic state (hypnosis) can be accurately and timely detected by more rapidly reacting than the current times.

The present invention may be applied to a medical instrument for evaluating an anesthetic depth and may also be applied to a brainwave signal processing-related instrument having a different signal processing technique.

### BRIEF DESCRIPTION OF THE DRAWINGS

FIG. 1 is a graph showing a change in brainwave according to an anesthetic degree.

FIG. 2 illustrates an apparatus for measuring an anesthetic depth by using a cepstrum technique in accordance with the present invention.

FIG. 3 is an algorithm of a method for measuring an anesthetic depth by using a cepstrum technique in accordance with the present invention.

FIG. 4 is a conceptual diagram of a mel filter bank illustrated in FIG. 2.

FIG. 5 is a conceptual diagram illustrating the vector calculation of a quantifying part illustrated in FIG. 2.

FIG. 6 shows measured results according to a conventional BIS and a method for measuring an anesthetic depth (MCI) in accordance with the present invention.

FIG. 7 is a drawing illustrating one example of a screen display part illustrated on FIG. 2.

FIG. 8 is a fisher score graph according to a selected filter of a mel filter bank.

### MODE FOR CARRYING OUT THE INVENTION

Hereinafter, specific embodiments for carrying out the present invention will be described with reference to the accompanying drawings. In the drawings, the dimensions of main parts are exaggerated and ancillary parts are omitted for clarity of illustration. Thus, the present invention should not be construed as limited to the drawings.

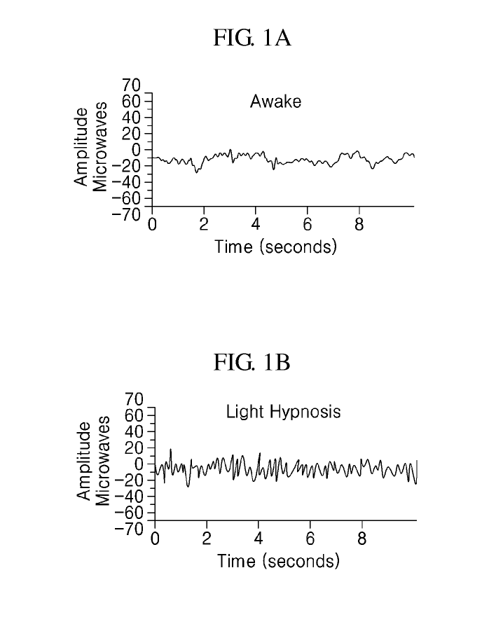

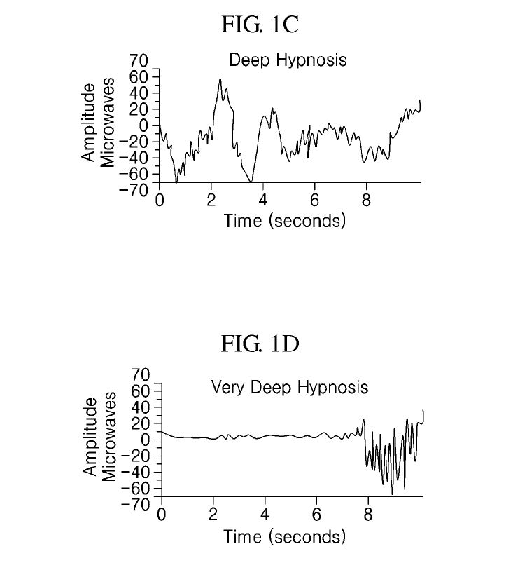

According to studies, it has been known that changes in characteristics of brainwaves during an operation have a great correlation with an anesthetic degree. Referring to FIG. 1, FIG. 1(a) shows measured brainwaves during an awake state, and brainwaves during an awake state have a small amplitude and a high frequency component. As a subject is entering into anesthesia (hypnosis), the amplitude becomes larger and the frequency component becomes lower as shown in FIGS. 1(b) and 1(c). When the subject is very deeply anesthetized, flat signals are outputted as shown in FIG. 1(d), and signals (burst suppression) having a high amplitude and a high frequency component are intermittently observed. Bio-signals such as changes in a heart rate, an electrocardiogram, and an electromyogram have a low direct correlation with an anesthetic degree. It is because various other reasons may affect a heart rate. On the other hand, unlike the correlation of a heart rate, it has been known through several researches that the characteristics of a brainwave signal have a direct correlation with an anesthetic degree of a patient when components of the brainwave signals are changed.

The present invention relates to an apparatus for accurately measuring an anesthetic degree of a patient from brainwaves, by a problem that a rapid change in an anesthetic state of a patient is not rapidly sensed due to a slow tracking speed which is a disadvantage of a conventional BIS apparatus has been solved, and accuracy has been improved.

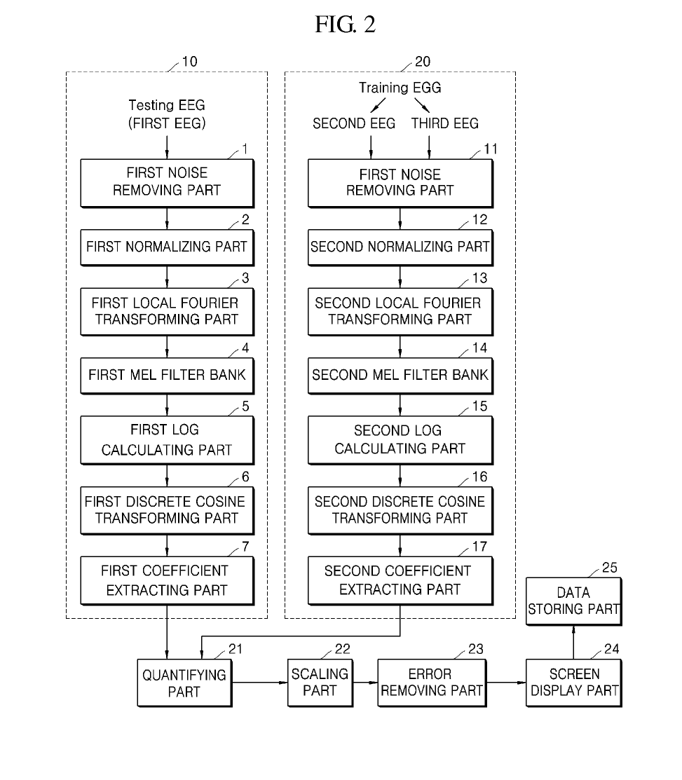

FIG. 2 is a drawing illustrating a structure of an apparatus for measuring an anesthetic degree by using a cepstrum technique in accordance with the present invention, and the apparatus for measuring an anesthetic degree by using a cepstrum technique illustrated in FIG. 2 includes a first feature vector extracting part 10, a second feature vector extracting part 20, a quantifying part 21, a scaling part 22, an error removing part 23, a screen display part 24, and a data storing part 25.

Main algorithms of the first feature and second vector extracting parts 10 and 20 are noise-removing and normalizing work and a mel frequency cepstral coefficient (hereinafter, referred to as “MFCC”) calculating technique. A mel frequency cepstrum (MFC) technique is one of the methods for extracting a power spectrum of a short section signal, which may be obtained by performing cosine transformation after a log power spectrum calculation is performed in a frequency domain of a nonlinear mel scale. The mel frequency cepstrum calculation equivalently divides a frequency band in a mel scale section through a mel filter bank. Through frequency warping to the mel scale section, an anesthetic depth may be accurately identified from an EEG signal, and application of an MFCC technique to measuring of an accurate anesthetic depth from brainwaves may result insignificant improvement in terms of fisher scoring and reaction speed, compared to a conventional BIS apparatus.

In particular, the first feature vector extracting part 10 includes a first noise removing part 1, a first normalizing part 2, a first local Fourier transforming part 3, a first mel filter bank 4, a first log calculating part 5, a first discrete cosine transforming part 6, and a first coefficient extracting part 7.

The first noise removing part 1 removes noise (artifact) caused by the eyes and noise caused by the movement of a subject, from an electroencephalography (hereinafter, referred to as “EEG”) signal (hereinafter, referred to as a “first EEG signal” to be distinguished from EEG signals in other states) of a patient through a patch and the like attached on the forehead and the like of the patient during an operation, and also removes noise through filtering by considering a signal of approximately 60 Hz or more as noise. Although information that may be obtained from the first EEG signal exists in various frequency bands, the first noise removing part 1 performs an analysis by using a frequency of approximately 0-60 Hz and considers the signal having a frequency of approximately 60 Hz or more as noise. The first noise removing part 1 performs at least one of, for example, a wavelet-based denoising technique or a low frequency band pass filtering technique.

Moreover, the first noise removing part 1 divides the first EEG signal or a signal with noise removed therefrom, which are serially inputted, into signals having a predetermined time unit (for example, 16 seconds). The divided signals may overlap signals adjacent thereto. For example, the divided signal overlaps a signal adjacent thereto for an interval of 15 seconds, and a divided signal is generated at every second to be outputted to the first normalizing part 2.

The first normalizing part 2 normalizes an output signal of the first noise removing part 1 as a root mean square (hereinafter, referred to as “RMS”) value.

The first local Fourier transforming part 3 divides an output signal of the first normalizing part 2 into sections by short time, and performs a Fourier transforming calculation for each of the sections to be summed up.

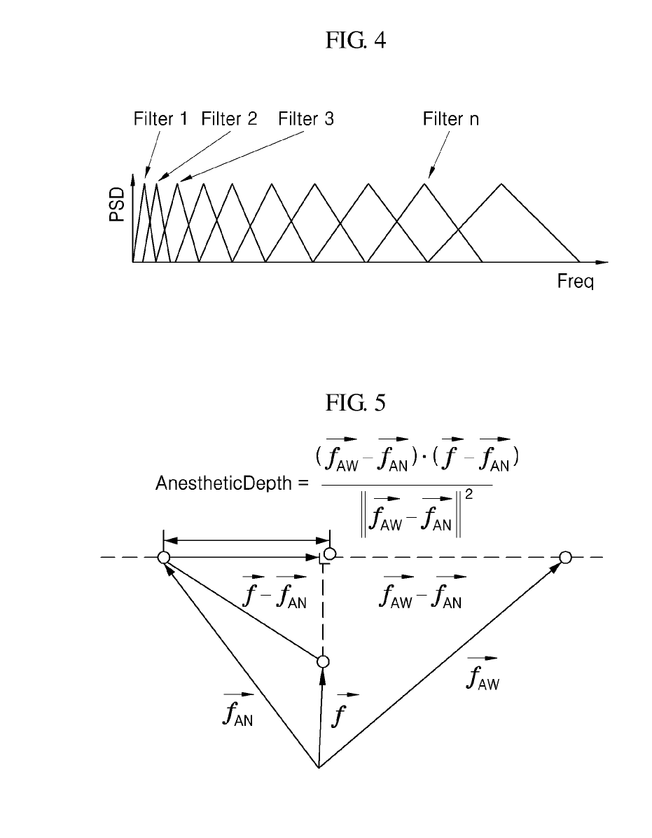

Referring to FIG. 4, the first mel filter bank 4 includes a plurality of filters (a first filter to an Nth filter), and in a frequency band of each filter, the filters overlap each other during predetermined sections and have different central frequencies. The first mel filter bank 4 receives an output signal of the first local Fourier transforming part 3 as an input signal and allows the received signal to be passed, and has a function in reducing a correlation. The central frequency of the first mel filter bank 4 is positioned in a bark or mel unit, and a bandwidth is determined according to a critical bandwidth. Because adjacent values of the first EEG signal have a high correlation with each other, the first EGG signal is made to pass through the first mel filter bank 4 to remove the correlation therebetween, and cepstral transformation is used. Even if noise is introduced, the first mel filter bank 4 may extract a more accurate value than a conventional BIS apparatus.

The first log calculating part 5 calculates an output of the first mel filter bank 4 on the log basis. The first log calculating part 5 may extract a more accurate value in a low frequency region and a high frequency region by means of log calculation.

The first discrete cosine transforming part 6 performs discrete cosine transformation (hereinafter, referred to as “DCT”) on an output signal of the first log calculating part 5. When discrete Fourier transformation (referred to as “DFT”) is used, signal power is concentrated on a high frequency band due to the non-continuity of a periodic signal. On the other hand, because DCT is continuous, a high frequency component is small, and signal power is thus concentrated on a low frequency band, thereby having an effect in accurately extracting an anesthetic depth. Compared to DFT, DCT has an effect in reducing signal distortion although filtering a high frequency signal which is smaller than a predetermined threshold.

The first coefficient extracting part 7 extracts a first feature vector by selecting a value passing through a predetermined filter (for example, a second filter of the plurality of filters) in the first mel filter bank 4 from an output of the first discrete cosine transforming part 6. Referring to FIG. 8, when a value (coefficient) passing through the second filter is selected and used, a fisher score is highest, and thus the first coefficient extracting part 7 extracts, as a first feature vector, the value passing through the second filter in the first mel filter bank 4.

Moreover, the second feature vector extracting part 20 includes a second noise removing part 11, a second normalizing part 12, a second local Fourier transforming part 13, a second mel filter bank 14, a second log calculating part 15, a second discrete cosine transforming part 16, and a second coefficient extracting part 17.

The structure of the second feature vector extracting part 20 is similar to the first feature vector extracting part 10. However, there is a difference in terms of training EEG signals as input signals. The functions of components are similar to those the first feature vector extracting part 10 as described above, and therefore description therefor will be replaced with the previous descriptions for the first feature vector extracting part 10. The training EEG signal includes a second EEG signal in a deep anesthetic state and a third EEG signal in an awake state (non-anesthetic state). The second feature vector extracting part 20 receives the second EEG signal as an input signal and extracts a second feature vector by performing noise removal, normalization, and an MFCC calculation. Also, the second feature vector extracting part 20 receives the third EEG signal as an input signal and extracts a third feature vector by performing noise removal, normalization, and an MFCC calculation. The second EEG signal and third EEG signal include sufficient clinical data.

The quantifying part 21 outputs an anesthetic depth index by dividing, into multiple sections, an area between the second feature vector and third feature vector which are set as both axes of a vector plane, and by quantifying a position at which the first feature vector is disposed, among the multiple sections. For example, as shown in FIG. 5, the quantifying part 21 compares the first feature vector (\vec{f}) with the second feature vector (\vec{f_{AN}}) and the third feature vector (\vec{f_{AW}}) to thereby determine whether the first feature vector (\vec{f}) is closer to the second feature vector (\vec{f_{AN}}) or the third feature vector (\vec{f_{AW}}) and then quantitatively calculate the compared result. Here, an anesthetic depth calculating method of the quantifying part 21 is the same as Equation 1. The first feature vector is \vec{f}, the second feature vector is \vec{f_{AN}}, and the third feature vector is \vec{f_{AW}}.

$$Anesthetic Depth = \frac{( \vec{{f}_{AW}} - \vec{{f}_{AN}} ) \cdot ( \vec{f} - \vec{{f}_{AN}} )}{\vert \vert \vec{{f}_{AW}} - \vec{{f}_{AN}} {\vert \vert}^{2}} \qquad ( Eq . 1 )$$

The scaling part 22 scales an output of the quantifying part 21 in an index ranging from 1 to 100.

The error removing part 23 removes abnormal signals from an output of the scaling part 22. It is highly likely that values resulted from noise are not correct values. A difference in a size from an adjacent point is represented as a histogram, and then points corresponding to top 0.5% are determined to be inappropriate values. For the inappropriate values, the predetermined number (for example, approximately 15-30) of past values is averaged, and calculation is performed by adding higher weighting to recent values.

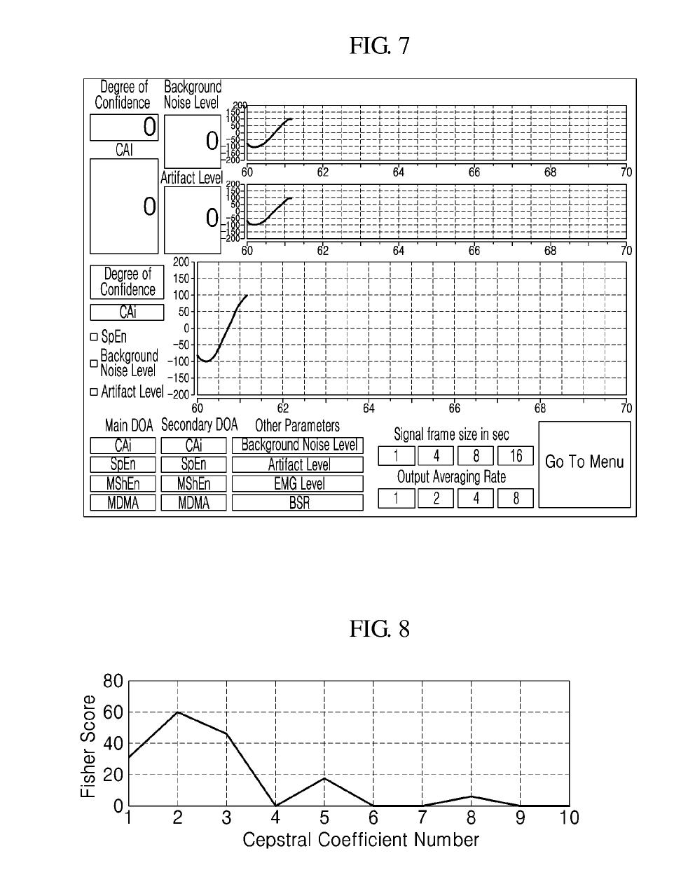

The screen display part 24, as illustrated in FIG. 7, displays an output of the scaling part 22 on a screen. When scaled signals are transmitted to monitoring software every second, the screen display part 24 displays an anesthetic depth on a screen by means of the monitoring software. At the same time, raw EEG signal and anesthetic depth index tendencies, signal quality, and other bio-signals (heart rate and electromyogram) are displayed together, thereby enabling an examiner to make an accurate determination.

The data storing part 25 stores measured anesthetic depth data, and the data may be extracted after an operation to be utilized as research materials in the future.

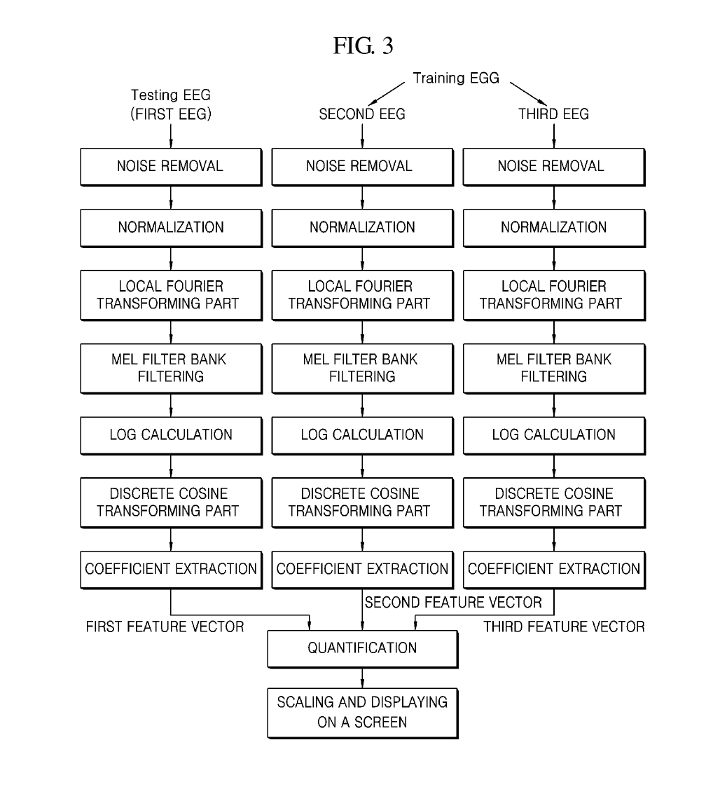

FIG. 3 illustrates an anesthetic depth measurement algorithm using a cepstrum technique in accordance with the present invention. Referring to FIG. 2, the anesthetic depth measurement algorithm using a cepstrum technique illustrated in FIG. 3 will be described.

The first noise removing part 1 removes and filters noise caused by the eyes and a frequency band of 60 Hz or more from the first EEG signal of patient during an operation.

Signals with noise removed therefrom have different sizes. Thus, the first normalizing part 2 normalizes the signals as RMS values, and the first local Fourier transforming part 3 divides the RMS values into section by short time and performs a Fourier transforming calculation thereon to be summed up.

The first mel filter bank 4 filters an output of the first local Fourier transforming part 3 through a plurality of filters in a mel scale frequency band, so as to reduce signal distortion by a frequency, and the first log calculating part 5 performs a log calculation on an output signal of the first mel filter bank 4.

The discrete cosine transforming part 6 performs discrete cosine transformation on an output of the first log calculating part 5, and the first coefficient extracting part 7 outputs a first feature vector by extracting only a signal, which passes through the second filter, from among outputs of the first discrete cosine transforming part 6.

Among training EEG signals, the second EEG signal which is an EEG signal in a deep anesthetic state also performs the same algorithm as the first EEG signal to output a second feature vector, and the third EEG signal which is an EEG signal in an awake state also performs the same algorithm as the first EEG signal to output a third feature vector.

The quantifying part 21 quantifies a section in which the first feature vector is positioned from the second feature vector and third feature vector, the scaling part 22 scales quantified signals with an index ranging from 1 to 100, and the screen display part 24 display the scaled result as an anesthetic depth index.

TABLE 1

| Algorithm | Fisher Score |
|---|---|
| MCI | 60.43 |
| BIS | 47.11 |

Referring to Table 1, when a concept of fisher scoring used in the field of pattern recognition is respectively applied to a conventional BIS apparatus and the present embodiment (MCI), the BIS apparatus has a fisher score of 47.11, and the MCI has a higher fisher score of 60.43. Fisher scoring means how well a state of a test signal can be classified when a signal to be tested is applied, and it can be seen that the apparatus for measuring an anesthetic depth according to the present invention provides a more accurate anesthetic depth than a conventional BIS.

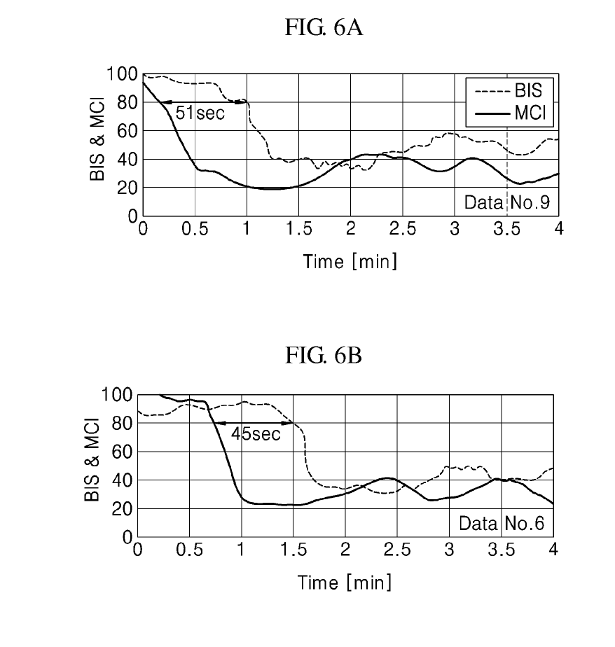

Referring to FIG. 6(a), it can be seen that the apparatus for measuring an anesthetic depth by using a cepstrum technique according to the present invention provides a tracking speed which is 51 seconds faster than that of a conventional BIS analyzing apparatus. Referring to FIG. 6(b), which shows another measurement result, it can be seen that the apparatus for measuring an anesthetic depth (MCI) according to the present invention provides a tracking speed which is 45 seconds faster than that of a conventional BIS analyzing apparatus. The apparatus for measuring an anesthetic depth according to the present invention can more accurately capture a change in a state during anesthesia by having a rapid reaction speed when an anesthetic degree is rapidly changed.

Although embodiments have been described with reference to a number of illustrative embodiments thereof, it should be understood that numerous other modifications and embodiments can be devised by those skilled in the art that will fall within the spirit and scope of the following claims.

1. A method for measuring an anesthetic depth by using a cepstrum technique, the method comprising:
extracting, by a first feature vector extracting part, a first feature vector by receiving a first EEG signal as an input signal and performing a mel frequency cepstral coefficient (MFCC) calculation;
extracting, by a second feature vector, a second feature vector and a third feature vector by receiving a second EEG signal in an anesthetic state and a third EEG signal in a non-anesthetic state as input signals and performing an MFCC calculation; and
outputting, by a quantifying part, an anesthetic depth index by dividing, into multiple sections, an area between the second and third feature vectors which are both axes of a vector plane, and quantifying a position, at which the first feature vector is located, among the multiple sections.

2. The method of claim 1, wherein the extracting of the first feature vector further comprises performing at least one of wavelet transformation or low frequency band pass filtering on the first EEG signal during an operation to remove noise and select and output a signal having only a predetermined frequency range.

3. The method of claim 1, wherein the extracting of the first feature vector comprises dividing the first EEG signal into sections by short time to perform Fourier transformation the divided signals for each section and then to sum up results.

4. The method of claim 3, wherein the extracting of the first feature vector comprises filtering the Fourier-transformed signals by a plurality of filter banks having different frequency bands and calculating a power spectrum for each of the signals.

5. The method of claim 4, wherein the extracting of the first feature vector comprises reducing signal distortion by a frequency by performing a log calculation on the power spectrum signals.

6. The method of claim 5, wherein the extracting of the first feature vector comprises extracting the first feature vector by performing discrete cosine transformation on the signals obtained after the log calculation and by selecting only a signal, which passes through a predetermined filter in the plurality of filter banks, from among the signals obtained after discrete cosine transformation.

7. An apparatus for measuring an anesthetic depth by using a cepstrum technique, the apparatus comprising:
a first feature vector extracting part configured to output a first feature vector by performing a mel frequency cepstral coefficient (MFCC) calculation on a first EEG signal;
a second feature vector extracting part configured to output a second feature vector and a third feature vector by performing an MFCC calculation on a second EEG signal in an anesthetic state and a third EEG signal in a non-anesthetic state; and
a quantifying part configured to output an anesthetic depth index by dividing, into multiple sections, an area between the second and third feature vectors which are both axes of a vector plane, and quantifying a position at which the first feature vector is located, among the multiple sections.

8. The apparatus of claim 7, wherein the first feature vector extracting part further comprises a noise removing part configured to perform wavelet transformation and low frequency band pass filtering on the first EEG signal.

9. The apparatus of claim 8, wherein the first feature vector extracting part further comprises a local Fourier transforming part which divides the first EEG signal into sections by short time to perform Fourier transformation on each of the sections.

10. The apparatus of claim 9, wherein the first feature vector extracting part further comprises a mel filter bank which includes a plurality of filters having different central frequencies and frequency bands overlapping each other for predetermined sections, and receives an output of the local Fourier transforming part as an input signal to filter the received signal.

11. The apparatus of claim 10, wherein the first feature vector extracting part further comprises a log calculating part configured to reduce signal distortion by a frequency by performing a log calculation on the signals filtered from the mel filter bank.

12. The apparatus of claim 11, wherein the first feature vector extracting part further comprises a discrete cosine transforming part configured to perform discrete cosine transformation on the signals obtained after the log calculation.

13. The apparatus of claim 12, wherein the first feature vector extracting part further comprises a coefficient extracting part configured to select only a signal, which passes through a predetermined filter in the filters of the mel filter bank, from among output signals of the discrete cosine transforming part, and output the selected signal to the first feature vector.

14. The apparatus of claim 13, further comprising an error removing part configured to represent the output of the first feature vector extracting part as a histogram and select a past output value for a signal outside an error range to be outputted as a weighted average value.

15. A computer-readable recording medium, in which a computer program for executing the method for measuring an anesthetic depth of claim 1 is stored.
